(19) United States
(12) Patent Application Publication
(10) Pub. No.: US 2020/0054231 A1
(43) Pub. Date: Feb. 20, 2020

(54) CARDIAC CYCLE-BASED ANALYZING SYSTEM, DEVICE AND METHOD

(71) Applicant: FAR EASTERN NEW CENTURY CORPORATION, Taipei City (TW)

(72) Inventors: Hsin-Kai LAI, Taipei City (TW); Yu-Chun WU, Taipei City (TW); Wei-Che HUNG, Taipei City (TW)

(21) Appl. No.: 16/426,710

(22) Filed: May 30, 2019

(30) Foreign Application Priority Data
Aug. 20, 2018 (TW) 107128985

(51) Int. Cl. A61B 5/04; A61B 5/0456; A61B 5/0472; A61B 5/00

(52) U.S. Cl. CPC A61B 5/04012; A61B 5/0456; A61B 5/0472; A61B 5/6804; A61B 5/681; A61B 5/6824; A61B 5/6823; A61B 5/7257; A61B 5/4809; A61B 5/4812; A61B 5/4815

(57) ABSTRACT

An analyzing method is to be implemented in an analyzing device, and includes: (A) dividing respective cycle lengths of a plurality of cardiac cycles of a user during sleep into a plurality of groups based on an order of occurrence of the cardiac cycles; (B) for each group, performing Fourier transform on the cycle lengths of the group to generate a respective spectrum; (C) for each group, calculating total power of the respective spectrum; and (D) determining, for each group, whether sleep quality of the user in a time period during which the corresponding cardiac cycles occurred is poor based on the total power of the respective spectrum and a predetermined threshold.

### CROSS-REFERENCE TO RELATED APPLICATION

This application claims priority of Taiwanese Patent Application No. 107128985, filed on Aug. 20, 2018.

### FIELD

The disclosure relates to cardiac cycle-based analysis, and more particularly to an analyzing system, an analyzing device and an analyzing method that perform analysis based on cardiac cycles.

### BACKGROUND

Electrocardiography (ECG) is a process of recording electrical activity of the heart of a person over a period of time using electrodes placed over the skin of the person. For a heart in normal sinus rhythm, the cardiac signal contained in the electrocardiogram includes a P wave, a QRS complex and a T wave sequentially in each cardiac cycle. The cardiac signal contained in the electrocardiogram can be analyzed and interpreted to diagnose various heart related diseases, such as left and right atrial abnormality, left and right ventricular hypertrophy, left and right bundle branch block, acute myocardial infarction, etc. Whether the cardiac signal has other applications is worth exploring.

### SUMMARY

Therefore, an object of the disclosure is to provide an analyzing system, an analyzing device and an analyzing method that perform analysis based on cardiac cycles and that can evaluate sleep quality.

According to an aspect of the disclosure, the analyzing system includes a sensor unit, a first processor unit, a second processor unit and a Fourier transform unit. The sensor unit is used to sense electrical activity of a heart of a user during sleep to generate a cardiac signal. The first processor unit is coupled to the sensor unit for receiving the cardiac signal therefrom, and calculates respective cycle lengths of at least some of a plurality of cardiac cycles of the user based on the cardiac signal. The Fourier transform unit is coupled to the second processor unit. The second processor unit is for receiving the cycle lengths, and divides the cycle lengths into a plurality of groups based on an order of occurrence of the at least some of the cardiac cycles. For each of the groups, the Fourier transform unit receives the cycle lengths of the group from the second processor unit, and performs Fourier transform on the cycle lengths of the group to generate a respective spectrum for the group. For each of the groups, the second processor unit receives the respective spectrum from the Fourier transform unit, calculates total power of the respective spectrum, and determines that sleep quality of the user in a time period during which the cardiac cycles corresponding to the cycle lengths of the group occurred is poor when the total power is smaller than a predetermined threshold, wherein the time periods respectively corresponding to the groups have a combined length equal to a combined length of the cardiac cycles.

According to another aspect of the disclosure, the analyzing device includes a processor unit, and a Fourier transform unit coupled to the processor unit. The processor unit divides respective cycle lengths of at least some of a plurality of cardiac cycles of a user during sleep into a plurality of groups based on an order of occurrence of the at least some of the cardiac cycles. For each of the groups, the Fourier transform unit receives the cycle lengths of the group from the processor unit, and performs Fourier transform on the cycle lengths of the group to generate a respective spectrum for the group. For each of the groups, the processor unit receives the respective spectrum from the Fourier transform unit, calculates total power of the respective spectrum, and determines that sleep quality of the user in a time period during which the cardiac cycles corresponding to the cycle lengths of the group occurred is poor when the total power is smaller than a predetermined threshold, wherein the time periods respectively corresponding to the groups have a combined length equal to a combined length of the cardiac cycles.

According to yet another aspect of the disclosure, the analyzing method is to be implemented in an analyzing device, and includes: (A) dividing respective cycle lengths of at least some of a plurality of cardiac cycles of a user during sleep into a plurality of groups based on an order of occurrence of the at least some of the cardiac cycles; (B) for each of the groups, performing Fourier transform on the cycle lengths of the group to generate a respective spectrum for the group; (C) with respect to each of the groups, calculating total power of the respective spectrum; and (D) determining, for each of the groups, whether sleep quality of the user in a time period during which the cardiac cycles corresponding to the cycle lengths of the group occurred is poor based on the total power of the respective spectrum and a predetermined threshold, wherein the time periods respectively corresponding to the groups have a combined length equal to a combined length of the cardiac cycles.

### BRIEF DESCRIPTION OF THE DRAWINGS

Other features and advantages of the disclosure will become apparent in the following detailed description of the embodiment with reference to the accompanying drawings, of which:

FIG. 1 is a block diagram illustrating an embodiment of an analyzing system according to the disclosure;

FIG. 2 is a flow chart illustrating an analyzing method performed by an analyzing device of the embodiment; and

FIG. 3 is a perspective view of a wearable device of the embodiment to be realized in the form of a smart garment.

### DETAILED DESCRIPTION

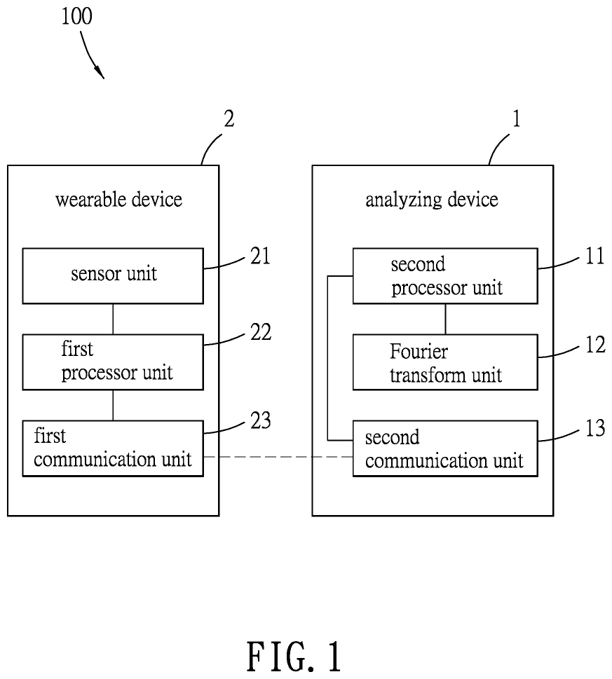

Referring to FIG. 1, an embodiment of an analyzing system 100 according to the disclosure includes a wearable device 2 and an analyzing device 1.

The wearable device 2 includes a sensor unit 21, a first processor unit 22 coupled to the sensor unit 21, and a first communication unit 23 coupled to the first processor unit 22. The sensor unit 21 is used to sense electrical activity of a heart of a user to generate a cardiac signal when the wearable device 2 is worn by the user. In this embodiment, the cardiac signal indicates respective R waves of a plurality of cardiac cycles at different times, but the disclosure is not limited thereto.

In a case where the wearable device 2 is in the form of a smart bracelet, the sensor unit 21 contacts a wrist of the user and senses pulses of the user to generate the cardiac signal when the wearable device 2 is worn by the user. Alternatively, in a case where the wearable device 2 is in the form of a smart garment, the sensor unit 21 contacts a chest of the user and senses heartbeats of the user to generate the cardiac signal when the wearable device 2 is worn by the user. However, the disclosure is not limited to the above examples.

The first processor unit 22 receives the cardiac signal from the sensor unit 21, and calculates respective cycle lengths of sampled ones of the cardiac cycles (hereinafter also referred to as “sampled cardiac cycles”) based on the cardiac signal. In this embodiment, the cycle length of a cardiac cycle is a length of an R-R interval between the R waves of adjacent two cardiac cycles, and is in the unit of ms. In addition, the first processor unit 22 further adjusts a magnitude of the cardiac signal based on respective peak values of the R waves. For example, the first processor unit 22 scales up the magnitude of the cardiac signal by a predetermined gain when the peak values of the R waves are smaller than a predetermined lower threshold (i.e., the heartbeats of the user are weak), and scales down the magnitude of the cardiac signal by another predetermined gain when the peak values of the R waves are greater than a predetermined upper threshold that is greater than the predetermined lower threshold (i.e., the heartbeats of the user are strong). Therefore, the first processor unit 22 can calculate the cycle lengths of the sampled cardiac cycles precisely regardless of whether the heartbeats of the user are weak or strong.

The analyzing device 1 includes a Fourier transform unit 12, a second processor unit 11 coupled to the Fourier transform unit 12, and a second communication unit 13 coupled to the second processor unit 11. The communication units 13, 23 comply with the same communication protocol, and are capable of establishing a communication link therebetween in a wired manner or in a wireless manner (e.g., using Wi-Fi, Bluetooth, etc.). Once the communication link is established, the analyzing device 1 and the wearable device 2 can transmit data therebetween.

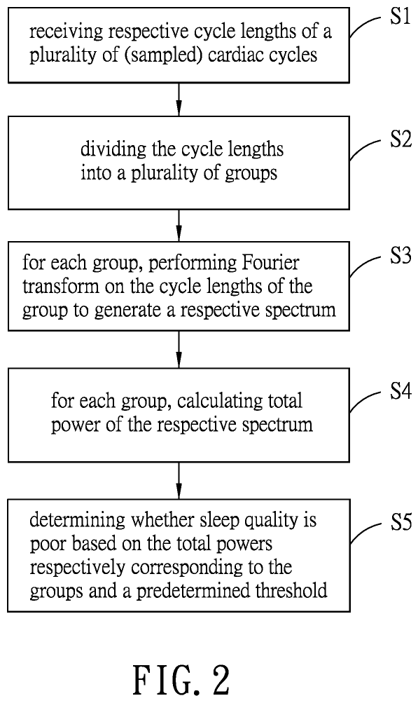

Referring to FIGS. 1 and 2, an analyzing method performed by the analyzing device 1 includes the following steps (S1-S5).

In step (S1), the second processor unit 11 receives the cycle lengths of the sampled cardiac cycles from the first processor unit 22 via the communication units 13, 23. In this embodiment, the cardiac signal is generated when the user is sleeping, and has a length of a first length value (e.g., four hours). In other words, the cardiac cycles have a combined length of the first length value.

In step (S2), the second processor unit 11 divides the cycle lengths into a plurality of groups based on the order of occurrence of the corresponding sampled cardiac cycles. In this embodiment, for each of the groups, a product of a total number of the cycle lengths in the group and a reciprocal of a sampling rate at which the corresponding sampled cardiac cycles are obtained equals a second length value (e.g., five minutes) smaller than the first length value.

In step (S3), for each of the groups, the Fourier transform unit 12 receives the cycle lengths of the group from the second processor unit 11, and performs Fourier transform (e.g., fast Fourier transform) on the received cycle lengths to generate a respective spectrum for the group.

In an example, the first processor unit 22 calculates the cycle length once every second (i.e., the sampling rate is one per second). For example, the cycle lengths of the cardiac cycles sampled at the first second, the second second, the third second and so on are respectively 1000 ms, 937.5 ms, 952 ms and . . . . Therefore, a total number of the cycle lengths per group is three hundred. In other words, for a person with a heart rate of over sixty beats per minute (e.g., seventy-two beats per minute), only the cycle lengths of sixty out of the seventy-two cardiac cycles will be calculated per minute in this example, and these sixty cardiac cycles are referred to as the sampled cardiac cycles. For each of the groups, the second processor unit 11 pads the three hundred cycle lengths of the group with two-hundred-and-twelve zeros to result in a sequence of five-hundred-and-twelve terms; and the Fourier transform unit 12 performs the fast Fourier transform on the five-hundred-and-twelve terms to generate the respective spectrum for the group.

In one embodiment, the first processor 21 calculates the cycle length of each cardiac cycle of the cardiac signal received thereby. In other words, all cardiac cycles are sampled cardiac cycles, and for each of the groups, a sum of all the cycle lengths in the group equals the second length value smaller than the first length value.

It should be noted that, in this embodiment, since the second length value is smaller than the first length value, the Fourier transform unit 12 has a relatively low calculation burden as compared to the Fourier transform unit in an example where the second length value is equal to the first length value. Of course, when the Fourier transform unit 12 has powerful calculation capability, the second length value may be equal to the first length value. In addition, padding the three hundred cycle lengths of each of the groups with the two-hundred-and-twelve zeros is to produce a sequence having five-hundred-and-twelve terms that meet the power-of-two requirements of the fast Fourier transform, but the disclosure is not limited to these values.

In step (S4), for each of the groups, the second processor unit 11 receives the respective spectrum from the Fourier transform unit 12, and calculates total power of the respective spectrum. In detail, the spectrum corresponding to an individual group has the following powers: ultra low frequency (ULF) power that is a sum of power components at frequencies lower than 0.003 Hz; very low frequency (VLF) power that is a sum of power components at frequencies from 0.003 Hz to 0.04 Hz; low frequency (LF) power that is a sum of power components at frequencies between 0.04 Hz and 0.15 Hz; and high frequency (HF) power that is a sum of power components at frequencies from 0.15 Hz to 0.4 Hz. The total power of the spectrum corresponding to an individual group is a sum of power components at frequencies lower than or equal to 0.4 Hz. In addition, the power component at a specific frequency is the square of an amplitude at the specific frequency, and each of the aforesaid powers is in the unit of ms².

In step (S5), for each of the groups, the second processor unit 11 determines whether sleep quality of the user in the time period which has a length of the second length value and during which the corresponding sampled cardiac cycles occurred is poor based on the total power corresponding to the group and a predetermined threshold. (In other words, the time periods respectively corresponding to the groups have a combined length of the first length value.) For each of the groups, the sleep quality of the user in the time period during which the corresponding sampled cardiac cycles occurred is determined to be poor when the total power corresponding to the group is smaller than the predetermined threshold. In this embodiment, the predetermined threshold is 4×10⁶ ms². The reasons for poor sleep quality include restless legs syndrome, sleep apnea, sleepwalking, frequent urination, having nightmares, etc.

It should be noted that, in this embodiment, the groups have the same total number of the cardiac cycles, and the time periods respectively corresponding to the groups have the same length. However, in other embodiments, the groups may have different total numbers of the cardiac cycles, and the time periods may have different lengths.

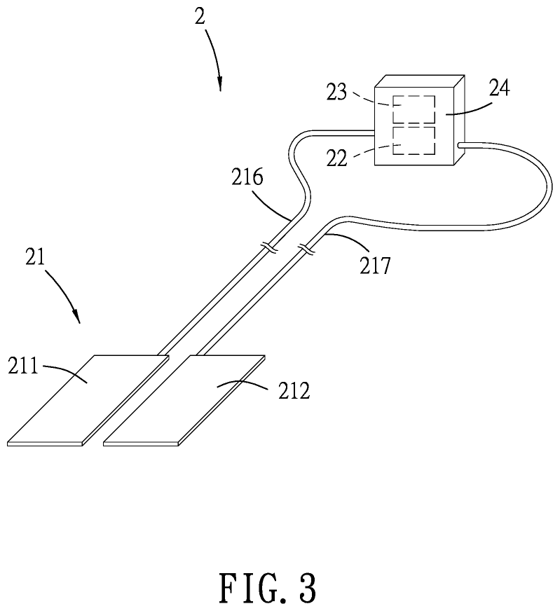

FIG. 3 illustrates the wearable device 2 to be implemented in the form of the smart garment. The smart garment further includes a housing 24 for accommodating the first processor unit 22 and the first communication unit 23. The sensor unit 21 includes two sensor elements 211, 212, and two conductive elements 216, 217 (e.g., conductive traces) each coupled between the first processor unit 22 and a respective one of the sensor elements 211, 212. Each of the sensor elements 211, 212 is, for example, a film-type physiology sensor. The film-type physiology sensor includes a fabric body, and a conductive coating layer embedded in the fabric body. The fabric body is, for example, a woven fabric (e.g., a plain woven fabric). The conductive coating layer includes a hydrophobic binder, and a plurality of conductive particles distributed in the hydrophobic binder. Materials that can be used for the hydrophobic binder include, but are not limited to, polyurethane (PU), siloxane, polyethylene terephthalate (PET), acrylic, etc. Materials that can be used for the conductive particles include non-metal materials, metal materials and any combination thereof. The non-metal materials include, but are not limited to, carbon nanotube (CNT), carbon black, carbon fiber, graphene and conductive polymers (e.g., poly(3,4-ethylenedioxythiophene) (PEDOT), polyacrylonitrile (PAN), etc.). The metal materials include, but are not limited to, gold, silver, copper and metal oxides (e.g., indium tin oxide (ITO), etc.).

It should be noted that, for convenience of illustration, FIG. 3 only depicts a portion of the smart garment without affecting the implementation of this embodiment. The portion of the smart garment depicted in FIG. 3 can be combined with a garment to meet application requirements, but the disclosure is not limited thereto. In addition, each of a total number of the sensor elements 211, 212 and a total number of the conductive elements 216, 217 is two in this embodiment, but may be three or more in other embodiments.

When the smart garment is worn by the user, one of the sensor elements 211, 212 (e.g., the sensor element 211) receives, from the chest of the user, a current signal that is generated due to the heartbeats of the user and that serves as the cardiac signal. The current signal flows to the first processor unit 22 via one of the conductive elements 216, 217 (e.g., the conductive element 216) that is coupled to said one of the sensor elements 211, 212 (i.e., the sensor element 211). The first processor unit 22 receives the current signal, converts the current signal into a voltage signal, and calculates the cycle lengths of the (sampled) cardiac cycles based on the voltage signal.

Referring back to FIG. 1, it should be noted that, in other embodiments, the following modifications may be made to this embodiment.

1. The first processor unit 22 is installed in the analyzing device 1, instead of the wearable device 2. In this case, the first communication unit 23 is coupled to the sensor unit 21, the first processor unit 22 is coupled between the second processor unit 11 and the second communication unit 13, the first processor unit 22 receives the cardiac signal from the sensor unit 21 via the communication units 13, 23, and the second processor unit 11 receives the cycle lengths of the (sampled) cardiac cycles directly from the first processor unit 22.

2. The first processor unit 22 is omitted. In this case, the first communication unit 23 is coupled to the sensor unit 21, and the second processor unit 11 receives the cardiac signal from the sensor unit 21 via the communication units 13, 23, and calculates the cycle lengths of the (sampled) cardiac cycles based on the cardiac signal.

It should also be noted that, in other embodiments, the following modification may be made to each of this embodiment and the aforesaid modified embodiments: the communication units 13, 23 are omitted. In this case, first, a storage unit (e.g., a memory card, a flash drive, etc.) is installed in the wearable device 2 to store the cardiac signal generated by the sensor unit 21 or the cycle lengths of the (sampled) cardiac cycles calculated by the first processor unit 22, and then is removed from the wearable device 2 to be installed in the analyzing device 1, so the first processor unit 22 of the analyzing device 1 receives the cardiac signal or the second processor unit 11 of the analyzing device 1 receives the cycle lengths of the (sampled) cardiac cycles from the storage unit.

In view of the above, by virtue of the analyzing device 1 that performs the analyzing method described above, the analyzing system 100 of this disclosure can determine, for each of the groups, whether the sleep quality of the user in the corresponding time period is poor (i.e., whether the user has high risk of sleep disorder). When the sleep quality of the user in at least one of the time periods, which respectively correspond to the groups, is determined to be poor, the user can control his/her weight by exercising regularly and having a balanced diet, so as to improve the sleep quality and reduce the risk of sleep disorder. Thereafter, if the sleep quality is still determined to be poor, the user can consult a medical specialist.

In the description above, for the purposes of explanation, numerous specific details have been set forth in order to provide a thorough understanding of the embodiment. It will be apparent, however, to one skilled in the art, that one or more other embodiments may be practiced without some of these specific details. It should also be appreciated that reference throughout this specification to “one embodiment,” “an embodiment,” an embodiment with an indication of an ordinal number and so forth means that a particular feature, structure, or characteristic may be included in the practice of the disclosure. It should be further appreciated that in the description, various features are sometimes grouped together in a single embodiment, figure, or description thereof for the purpose of streamlining the disclosure and aiding in the understanding of various inventive aspects.

While the disclosure has been described in connection with what is considered the exemplary embodiment, it is understood that the disclosure is not limited to the disclosed embodiment but is intended to cover various arrangements included within the spirit and scope of the broadest interpretation so as to encompass all such modifications and equivalent arrangements.

What is claimed is:

1. An analyzing system comprising:
a sensor unit used to sense electrical activity of a heart of a user during sleep to generate a cardiac signal;
a first processor unit coupled to said sensor unit for receiving the cardiac signal therefrom, and calculating respective cycle lengths of at least some of a plurality of cardiac cycles of the user based on the cardiac signal;
a second processor unit; and
a Fourier transform unit coupled to said second processor unit;
said second processor unit for receiving the cycle lengths, and dividing the cycle lengths into a plurality of groups based on an order of occurrence of the at least some of the cardiac cycles;
for each of the groups, said Fourier transform unit receiving the cycle lengths of the group from said second processor unit, and performing Fourier transform on the cycle lengths of the group to generate a respective spectrum for the group;
for each of the groups, said second processor unit receiving the respective spectrum from said Fourier transform unit, calculating total power of the respective spectrum, and determining that sleep quality of the user in a time period during which the cardiac cycles corresponding to the cycle lengths of the group occurred is poor when the total power is smaller than a predetermined threshold, wherein the time periods respectively corresponding to the groups have a combined length equal to a combined length of the cardiac cycles.

2. The analyzing system of claim 1, wherein the predetermined threshold is 4×10⁶ ms².

3. The analyzing system of claim 1, wherein the total power of the spectrum is a sum of power components at frequencies lower than or equal to 0.4 Hz.

4. An analyzing device comprising:
a processor unit; and
a Fourier transform unit coupled to said processor unit;
said processor unit dividing respective cycle lengths of at least some of a plurality of cardiac cycles of a user during sleep into a plurality of groups based on an order of occurrence of the at least some of the cardiac cycles;
for each of the groups, said Fourier transform unit receiving the cycle lengths of the group from said processor unit, and performing Fourier transform on the cycle lengths of the group to generate a respective spectrum for the group;
for each of the groups, said processor unit receiving the respective spectrum from said Fourier transform unit, calculating total power of the respective spectrum, and determining that sleep quality of the user in a time period during which the cardiac cycles corresponding to the cycle lengths of the group occurred is poor when the total power is smaller than a predetermined threshold, wherein the time periods respectively corresponding to the groups have a combined length equal to a combined length of the cardiac cycles.

5. The analyzing device of claim 4, wherein the predetermined threshold is 4×10⁶ ms².

6. The analyzing device of claim 4, wherein the total power of the spectrum is a sum of power components at frequencies lower than or equal to 0.4 Hz.

7. The analyzing device of claim 4, wherein said processor unit is used to receive the cycle lengths.

8. The analyzing device of claim 4, wherein said processor unit is used to receive a cardiac signal related to electrical activity of a heart of the user, and calculates the cycle lengths based on the cardiac signal.

9. An analyzing method to be implemented in an analyzing device, said analyzing method comprising:
(A) dividing respective cycle lengths of at least some of a plurality of cardiac cycles of a user during sleep into a plurality of groups based on an order of occurrence of the at least some of the cardiac cycles;
(B) for each of the groups, performing Fourier transform on the cycle lengths of the group to generate a respective spectrum for the group;
(C) with respect to each of the groups, calculating total power of the respective spectrum; and
(D) determining, for each of the groups, whether sleep quality of the user in a time period during which the cardiac cycles corresponding to the cycle lengths of the group occurred is poor based on the total power of the respective spectrum and a predetermined threshold, wherein the time periods respectively corresponding to the groups have a combined length equal to a combined length of the cardiac cycles.

10. The analyzing method of claim 9, wherein the predetermined threshold is 4×10⁶ ms².

11. The analyzing method of claim 9, wherein the total power of the spectrum is a sum of power components at frequencies lower than or equal to 0.4 Hz.
